(19) United States
(12) Patent Application Publication
(10) Pub. No.: US 2002/0112261 A1
(43) Pub. Date: Aug. 15, 2002

(54) Transformation of monocotyledoneous plants using Agrobacterium

(76) Inventors: Brenda Rojas, Guelph (CA); Stephen R. Bowley, Cambridge (CA); Alissa Devereaux, Guelph (CA)

Correspondence Address: Kathy Smith Dias, Esq., Heslin Rothenberg Farley & Mesiti P.C., 5 Columbia Circle, Albany, NY, 12203, US

(21) Appl. No.: 09/965,663

(22) Filed: Sep. 26, 2001

Related U.S. Application Data
(63) Continuation of application No. PCT/CA00/00306, filed on Mar. 24, 2000.

(30) Foreign Application Priority Data
Mar. 26, 1999 (CA) 2,267,014

(51) Int. Cl. A01H 5/00; C12N 1/21

(52) U.S. Cl. 800/294; 435/252.2

(57) ABSTRACT

The present method is directed to a method for the transformation of a monocot plant comprising exposing explant tissue of said monocot to an Agrobacterium strain under vacuum in the presence of a phenolic compound, said Agrobacterium strain comprising a heterologous gene of interest within a vector. The Agrobacterium is removed from the explant tissue, and an antibiotic against the Agrobacterium is added, and the transformed tissue is plated onto growth medium, grown, and then plated onto selection media. The method may be used with vectors comprising either a vir+ vector, or a super virulent vector.

### BACKGROUND OF THE INVENTION

The host range of Agrobacterium spp. is typically restricted to dicotyledons such as alfalfa, cotton, tobacco, etc. As monocots are not naturally parasitized by Agrobacterium, transformation of monocot species via Agrobacterium has been problematic. For example, U.S. Pat. Nos. 5,780,709, 5,780,708, 5.508,468 and Potrykus (1990, Bio/Technol 8;535-542; 1990, Physiol. Plant 79:125-134) comment on the unreliability of Agrobacterium for maize transformation protocols.

Many Agrobacterium transformation methods used for monocots require injury to The tissue, either by wounding tissue in some manner or particle bombardment. For example, U.S. Pat. Nos. 5.187,073 and 5,177.010 disclose a method for transforming monocots by wounding a seedling from the scutellar node to about the coleopule node, and inoculating the wound with a vir+ strain of Agrobactecterium rumefaciens. U.S. Pat. Nos. 5,712,135 and 5.641.664 disclose the wounding of embryogenic callus by, for example, cutting up the callus into pieces, and transferring DNA into the wounded tissue. The use of microparticle bombardment of callus cultures is disclosed in U.S. Pat. Nos. 5,790,709; 5,780,708: 5,773,269: 5.554,798; 5,508.468; 5,484.956 and 5.405.765. Similarly microparticle bombardment of suspension cultures is taught in U.S. Pat. Nos. 5,550,318 and 5.489,520 Treatment of callus cultures with a wall degrading enzyme, thereby wounding the tissue is described in U.S. Pat. No. 5,712.135. Similarly, e use of an inhibitor of poly-(ADP-ribose) polymerase activity (niacinamide) for reducing the stress response in cultured tissue in order to permit transformation is disclosed in WO97/06267.

Prior art methods for transforming monocots requires the use of super virulent vectors (e.g. U.S. Pat. Nos. 5,712,1355 5,641,664; 5,773,269; 5,554,798; 5,484,956, 5,405,765; and 5,591,616) that exhibit extremely high transformation efficiencies when used in dicot transformation, A super virulent vector comprises the vir region from a super virulent Agrobacterium, such as Agrobacterium rumefaciens A281 (e.g. U.S. Pat. No. 5,591,616). A protocol for monocot transformation using a super virulent vector, in the absence of explant wounding is found in U.S. Pat. No. 5,591,616 (see also Hiei et al 4. 1997, Plant. Molec. Biol. 35,205-218). The use of super virulent vectors in the disclosed protocol resulted in 95-100% transformation efficiency. However, the use of supervirulent vectors is associated with several problems including the difficulty of removing the Agrobacterium following exposure to the explant tissue, and a significant increase in the cost of the transformation protocol.

There is therefore a need within the art to transform monocot plants with reliable and simplified protocols using Agrobacterium mediated transformation protocols that incorporate readily available vectors.

It is an object of the invention to overcome disadvantages of the prior are.

The above object is met by the combinations of features of the main claims, the sub-claims disclose further advantageous embodiments of the invention.

### SUMMARY OF THE INVENTION OF THE

The present invention relates To a method for we transformation of monocot plants. More specifically This invention relates to a method for the transformation of monocot plants using Agrobacterium sp.

According to the present invention there is provided a method (A) for the transformation of a monocot plant comprising,

i) exposing explant Tissue of the monocot to an Agrobacterium strain under vacuum in the presence of a phenolic compound, the Agrobacterium strain comprising a heterologous gene of interest within a vector;

ii) removing the Agrobacterium from the explant tissue;

iii) adding an antibiotic against the Agrobacterium; and

iv) selecting explant issue.

This invention also relates to the method (A) as defined above wherein the step of selecting explant tissue further comprises:

i) maintaining the explant tissue on media in the absence of a selection agent in order for the tissue to differentiate, thereby producing differentiated calli;

ii) transferring the differentiated calli to media containing a selection agent; and

iii) obtaining calli that grow in the presence of the selection agent thereby obtaining transformed monocot calli.

This invention is also directed to the method (A) as defined above wherein the Agrobacterium comprises a regular binary, or a super virulent vector.

This invention also relates To a method (A) as defined above wherein the explant tissue comprises a callused coleoptile node, or comprises a zygotic embryo.

This invention is also directed w a method (B) for the transformation of a monocot plant comprising;

i) transferring explant tissue of the monocot plant to a suspension of Agrobacterium to obtain a mixture the Agrobacterium strain comprising a heterologous gene of interest within a vector;

ii) maintaining the mixture under vacuum in the presence of acetosyringone;

iii) releasing the vacuum and further incubating the explant tissue in the presence of said Agrobacterium;

iv) transferring the explant tissue into fresh media comprising acetosyringone and incubating in the dark:

v) washing the explant tissue with an antibiotic against the Agrobacterium, transferring said explant tissue to fresh media and allowing the explant tissue to differentiate, thereby producing differentiated calli;

Vi) transferring the differentiated calli to media containing a selection agent, and maintaining he differentiated calli in the light; and

vii) obtaining calli that grow in the presence of the selection agent.

The present invention also embraces the method (B) as defined above wherein the Agrobacterium comprises a regular binary, or a super virulent vector.

This invention also pertains to the method (B) as defined above wherein the fresh media of step v) comprises an antibiotic against said Agrobacterium.

The present invention relates to the method (B) as defined above wherein the explant tissue is callused coleoptile node, or comprises a zygotic embryo.

The present invention also pertains to a method (C) for the transformation of a monocot plant comprising,

i) transferring explant tissue from the monocot plant into media comprising a phenolic compound and a suspension of Agrobacterium to obtain a mixture, the Agrobacterium strain comprising a heterologous gene of interest within a vector;

ii) washing The explant tissue with an antibiotic against the Agrobacterium and transferring the explant tissue to fresh media comprising acetosyringone and incubating The explant tissue in ;he dark;

iii) transferring the explant tissue to fresh media and allowing the explant tissue to differentiate, thereby producing differentiated calli;

iv) transferring the differentiated calli to media containing a selection agent, and maintaining the differentiated calli in the light, and

v) obtaining calli that grow in the presence of the selection agent.

The present invention pertains to a method for the transformation of monocot plants with a gene of interest, wherein no wounding of the tissue is required for transformation to occur. Furthermore, monocot plants may be transformed using a regular binary vector following the method as described herein, so that the use of super virulent vectors is not required. However, the present method may also be used with super virulent vectors. Without wishing to be bound by theory, the success of transformation of monocot plants using e method of the present invention may in part due to the treatment of explant tissue with Agrobacterium in liquid culture, and in part to the selection of transformed calli following differentiation of the explant tissue. Furthermore, the step of vacuum infiltration may also be used to increase the rate of transformation.

This summary of the invention does not necessarily describe all necessary features of the invention but that the invention may also reside in a sub-combination of the described features.

### BRIEF DESCRIPTION OF THE DRAWINGS

These and other features of the invention will become more apparent from the following description in which reference is made to The appended drawings wherein:

FIG. 1 shows a T-DNA construct 35S-mnSOD; Ubi-bar comprising a maize intron FIG. 2 shows PCR analysis for the bar and MnSOD genes FIG. 2(A) analysis of bar gene. FIG. 2(13), analysis of MnSOD gene. Lane1:1 Kb DNA ladder; Lanes 2 to 14: plant DNA from 13 independent transgenic creeping bentgrass plants; Lanes 15 to 18: DNA from four non-transgenic creeping bentgrass plans; Lane 19: no DNA; Lane 20pSOD-bar plasmid DNA.

FIG. 3 shows PCR results of putative transgenic perennial ryegrass plants using primers to amplify the bar gene. L=1 kB ladder, +=pSODbar plasmid (Lanes 17A,19A),−=H20 negative control (Lanes 21A, 23A; FIG. 3A), nt=non-transgenic perennial ryegrass plane negative control (Lanes 19B, 21B, 23B; FIG. 3B). In FIGS. 3A and 3B, 35 of the 39 extracts amplified the expected PCR product.

FIG. 4 shows Southern hybridization of DNA obtained from 5 independent transgenic plants digested with BstXI and probed with a bar gene probe, Arrow indicates 5000 bp fragment Lane 1—Ladder. Lane 2-6—independent transgenic plants Lane 7—pSODbar plasmid

FIG. 5 shows Western analysis of perennial ryegrass transformed as described herein. Lane 1,2 Non transgenic plants, Lane 3-6—independent transgenic plants. Arrow indicates unique protein of approximately 90 kDa molecular weight present in lanes 3 (plant E50), 4 (plant E68), 6 (plant E101).

FIG. 6 shows a native protein isozyme gel of proteins obtained from transformed plants. In order to stain specifically for MnSOD activity, Cu/ZnSOD and FeSOD isozymes were inactivated by 30 min. incubation at room temperature in 3% H₂O₂ in phosphate buffer, followed by staining for SOD activity as described (Example 2). Lane 1—MnSOD marker from E coli. Lane 2—Non transgenic plant. Lane 3,4—independent transgenic plants E50 and E68. Arrow indicates MnSOD activity

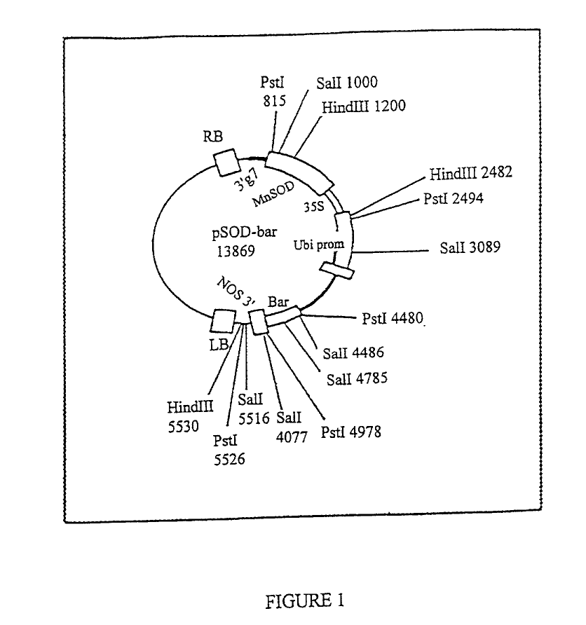

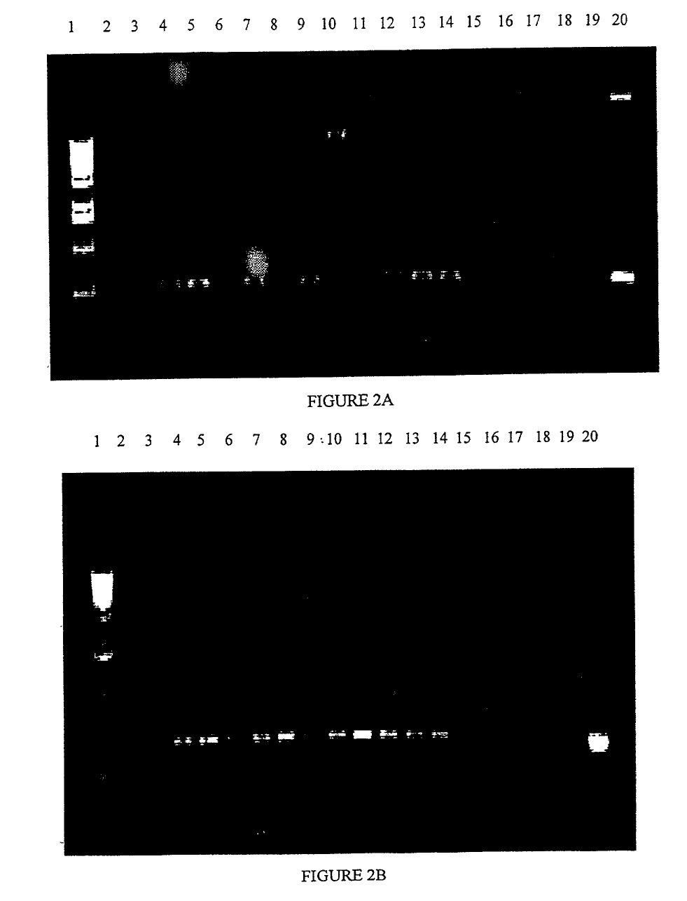

### DESCRIPTION OF PREFERRED EMBODIMENT

The present invention relates to a method for the transformation of monocot plants. More specifically this invention relates to a method for the transformation of monocot plants using Agrobacterium sp.

The following description is of a preferred embodiment by way of example only and without limitation to the combination of features necessary for carrying the invention into effect.

The method of the present invention is directed to The transformation of monocot plants, using regular Agrobacterium-based vectors. By regular binary vector, it is meant vectors tat result in an average transformation efficiency when used in dicot transformation systems, for example, but not limited to, Bin19. Such vectors may be used within a variety of Agrobacterium rumefaciens strains, for example, but not lilted to, Agrobacterium strain C58.

Furthermore, the transformation protocol of the present invention does not require any wounding of tissue in order To achieve cellular uptake of the vector.

The method of the present invention involves the preparation of explant tissue, which includes but is not limited to the coleoptile node, or zygotic embryo and exposure of this tissue to a suspension of Agrobacterium cells comprising a gene of interest (within an optional marker gene) within a vector, to be introduced within the monocot plant. If coleoptile node tissue is used, then preferably the tissue is callused. However, it is to be understood that other plant tissues or explant tissues may also be used for transformation following the methods as described here. The Agrobacterium suspension—explant tissue mixture may be maintained for a period of time under vacuum at a temperature from about 15° C. to about 28° C. Preferably, the mixture is incubated at about 22° C.

The vacuum treatment of the mixture may last from about 1 to about 120 minutes, preferably, the incubation rime under vacuum is from about 5 to about 60 minutes. More preferably, the incubation under vacuum is from about 10 to about 15 minutes. Without wishing to be bound by theory it is thought that the contact between the cells of the explant and Agrobacterium, is enhanced under vacuum An approximately two fold increase in transformation efficiency is obtained by vacuum creating explants, when compared with an analogous protocol that lacks the vacuum infiltration step (see Example 1). Following the vacuum treatment, the explants remain Within The Agrobacterium suspension for a period of time, for example, from 1 to about 60 minutes, however, longer incubation Times may also be used if desired

The Agrobacterium suspension comprises any incubation medium suitable for the culture of the explant. For example, such a medium would include, but is nor limited to a basal medium, for example MS (Murashige and Skoog basal medium (1962, Physiol. Plant 15:473-497) further comprising;

sucrose from about 5 g/L to about 50 g/L;

at least one amino acid, such as but nor limited to, asparagine, from about 50 mg/L to about 300 mg/L,

2,4-D, or its equivalent, from about 1 mg/L to about 50 mg/L, BAP, or its equivalent, from about 1 mg/L to about 50 mg/L, or both 2,4-D and BAP, from about 1 mag/l to about 50 mg/L; and optionally,

casein hydrolysate (from about 50 mg/l to about 1 g/L), 2,4-D (from about 0.1 mg/L to about 5 mg/L), dicamba (from about 0.5 mg/l to about 20 mg/l), and a phenolic, for example, but not limited to, acetosyringone (from about 5 μM to about 200 μM).

It is preferred chat this medium also comprise a phenolic compound capable of inducing the vir genes on Ti plasmid, for example, but not limited to, acetosyringone. The medium is at a pH from about 4.5 to about 7, preferably at a pH of about 5.2 to about 5.8. Examples of several media, which are to be considered non-limiting, are presented in Table 1.

TABLE 2
Tissue Culture Media Composition

| | Embryo Induction | | Co-cultivation | | Embryo Dev |
|---|---|---|---|---|---|
| | Medium (solid) | | Medium (liquid) | | Medium |
| | MSCB | MSPR | MSCBcc | MSPRcc | MSO |
| pH | 5.8 | 5.8 | 5.2 | 5.2 | 5.8 |
| Basal medium | MS | MS | MS | MS | MS |
| Sucrose (g/L) | 20 | 20 | 20 | 20 | 10 |
| asparagine (mg/L) | 150 | 150 | 150 | 150 | — |
| casein hydrolysate | — | 500 | — | 500 | — |
| (mg/L) | | | | | |
| BAP (mg/L) | 10 | 10 | 10 | 10 | — |
| 2,4-D (mg/L) | 0.5 | — | 0.5 | — | — |
| dicamba (mg/L) | — | 5 | — | 5 | — |
| Phytagel (gfL) | 2 | 2 | — | — | 2 |
| acetosyringone (μM) | — | — | 40 | 40 | — |

Following transformation, the explants are permitted to differentiate on a suitable media, for example but nor limited to MSCB or MSPR (Table 1), from about 2 weeks to about 3 months, prior to selection for the occurrence of the selectable marker to produce differentiated calli. Preferably the period of rime for producing differentiated calli is form about 4 to about 8 weeks (see Table 2, Example 1)

Following differentiation, calli are transferred to a medium, such as MSO, which contains an appropriate selection agent. To aid in identification of transformed plant cells, the constructs of this invention may be manipulated to include plant selectable markers. Useful selectable markers include enzymes which provide for resistance to an antibiotic such as, but not limited to, phosphinitricin, gentamycin, hygromycin, kanamycin. Similarly, enzymes providing for production of a compound identifiable by colour change such as GUS (β-glucuronidase), or luminescence, such as luciferase may also be employed. The step of selecting for occurrence of the selectable marker may be repeated using higher concentrations of selection agent as required. Performing the step of allowing callus growth following co-cultivation or tissue with Agrobacterium prior o transfer to selection medium results in higher rates of transformation.

The protocol as described herein may be used for the transformation of any monocotyleonous plant, including for example, but rot limited to, grasses. for example, Kentucky bluegrass (Poa prarensis), Canada bluegrass (Poa compressa) and bromgrass (Bromus inermis), agricultural plants, for example corn (Zea maize), rice, and wheat.

The above description is not intended to limit the claimed invention in any manner, furthermore, the discussed combination of features might not be absolutely necessary for the inventive solution

The present invention will be further illustrated in the following examples. However it is to be understood that these examples are for illustrative purposes only, and should not be used to limit scope of the present invention in any manner.

### EXAMPLES

Transformation Protocol

Seeds are surface sterilized and germinated for 3 days in the dark, The seedlings are transferred to solid embryogenic callus induction medium (e.g. MSCB or MSPR, depending upon the species used) and maintained in the dark for 10-60 days at 22-25° C. Cultured explant tissue, including the callused coleoptile node or mature zygotic embryo is excised and incubated for 1 hour on liquid embryogenic callus induction medium (e.g. MSCBcc or MSPRcc). A suspension of Agrobacterium (strain C58; density OD 0.5-0.7) is also incubated for 1 hour on liquid embryogenic callus induction medium. Cultured explant tissue is transferred to a suspension of Agrobacterium (strain C58; density OD 0.5-0.7) for 1 hour comprising liquid embryogenic callus induction medium (e. MSCBcc or MCPRcc) and acetosyringone. For the first 10-15 mm, tubes containing the Agrobacterium—explant tissue mixture are maintained under vacuum (about 18-24 mm Hg; 24-32 mbar). The mixture is then maintained at atmospheric pressure at 22° C. for the following 45 min.

The explants arc transferred c o a container containing liquid embryogenic callus induction medium (e.g. MSCBcc or MSPRcc) comprising acetosyringone (pH 5.2) and maintained for 3 days in die dark at from about 22 to about 25° C. The callused nodes are removed, washed for 0.5 hr with an antibiotic (for example claforan, 2 g/l) to Kill Agrobacterium, thereby killing remaining Agrobacterium, and blotted dry on sterile filter paper.

The explants are Then placed on solid embryo induction medium (e.g. MSCB or MSPR) containing an antibiotic to eliminate Agrobacterium, for example, claforan (300 mg/L) and cultured in the dark from about 22° C. to about 25° C. After about 1-2 months calli start to differentiate and sections start to separate, the calli are transferred to MSO medium (see Table 1 for composition), containing a selectable marker selection agent, and cultured in light. The concentration of the selection agent is increased over time in order to select Transformed plants. The step wise increase in selection agent concentration avoids toxic effects caused by death of large numbers of tissue/cells during the selection process.

Construction of Plasmid.

The operations for constructing the plasmid were carried out in accordance with Ausubel, F. M., Brent, R. Kingston, R. F., Moore, D. D., Seidman, J. G., Smith, J. A. and Struhl, K. 1989. Current protocols in molecular biology. John Wiley & Sons, NY

The pNOS-nptII region (2349 bp) was deleted from PEX1SOD (Bowler et al, 1991. Embo J. 10:1723-1732) using the restriction enzymes SruI and NruI The Ubiquitin promoter-intron-Bar-Nos 3′ region from the pAHC25 vector (Christensen, A. H. and Quail, P. H. 1996. Transgenic Research 5:213-18) was excised using partial digestion With the restriction enzyme EcoRI. Following electrophoretic separation, The 2890 bp fragment was filled in with Klenow fragment enzyme (Gibco BRL), and inserted into pEX1SOD at he site previously occupied by pNOS-nptII. Three sets of digestion with the restriction enzymes PstI, HindIII and Sall followed by electrophoretic separation confirmed the insertion orientation of The 2890 bp fragment. The obtained plasmid was named pSOD-bar.

Introduction of pSOD-bar into Agrobacterium

Competent Agrobacterium rumefaciens C58Cl cells, which contained the virulent plasmid pMP90, were produced as described by Ausbel et al. (Ausubel, F. M., Brent, R., Kingston, R. E., Moore, D. D., Seidman, J. G., Smith, J. A. and Struhl, K 1989. Current protocols in molecular biology. John Wiley & Sons, NY). The pSOD-bar vector was elecroporated into cells using a Gibco BRL Cell-Porator Electroporation System in accordance with the manufacturer's instructions. 20 ng of the pSOD-bar plasmid were used to transform 20 μl of competent A.tumefaciens C58Cl cells. After electroporation and removal of the sample from the microelectroporation chamber, the sample was incubated for 2 h at 28° C. in 1 ml of SOC medium. After 1 h incubation, a 100μL aliquot was plated on solid LB medium containing the selection antibiotics Streptomycin (30 mg/l) and Spectinotmycin (30 mg/L) and incubated in the dark for 48 hrs at 28° C. Long term cultures for use in transformation experiments were produced as described by Ausubel et at. (1989).

### Example 1

Transformation of Creeping Bentgrass

Creeping bentgrass (Agrostis palustris) cv ‘Cobra’ was transformed as described above with pSOD-bar. To obtain cultured explant tissue, seeds were surface sterilized, germinated for 3 days in the dark, and seedlings transferred to solid embryogenic callus induction medium (MSCB) and maintained in the dark for 3-5 wks at 22-25° C. The construct was introduced via Agrobacterium mediated transformation s outlined above using MSCBcc (liquid) cultivation medium Following the 3 day co-cultivation, the explants were then placed on MSCB solid medium containing 300-500 mg/L claforan and cultured in the dark from about 22 to about 25° C. After 4-8 wks on MSCB medium in the dark, transformed calli were transferred to MSO medium containing the selection agent PPT (phosphinothricin) at 5 mg/L and claforan (300 mg/L) and cultured in the light to allow the plants to grow. After 4 wk, surviving calli were transferred to fresh MSO medium containing 5 mg/L PPT and claforan (300 mg/L), and after an additional 2 wk, were transferred to MSO medium containing 10 mg/L PPT.

Plants recovered from this process were sampled for the presence of the introduced genes using PCR. Leaf tissue (approx 0.1 g) was ground with liquid nitrogen and placed into a 1.5 ml microcentrifuge tube containing 600 μL of warm (55° C.) 2×CTAB extraction buffer (Rogers and Bendich, 1994; Plant Molecular Biology Manual D1, Kluwer Academic Publishers, Belgium). Tubes were incubated for 15-20 min at 55° C., cooled to room temperature, 300 μL of chloroform/octanol (24;1) are added, and mixed by inversion for 5 min. Tubes were centrifuged (13,000×g) for 5 min at room temperature, and the supernatant transferred to a new microcentrifuge tube. 300 μL of chloroform/octanol (24:1) was added an the mixing, centrifugation, and the supernatant transfer steps were repeated. 5 μL Rnase A (10 mg/L) was added, the cubes were inverted 2-3 times to mix the solution and left for 15 min at room temperature. After 15 min. 600 ,μL of isopropanol was added, the rules were mixed by inverting 2-3 times and held at −20° C. for at least 2 hrs. The rubes were centrifuged 15-20 min at4° C. and the supernatant was discarded. The DNA pellet was washed in 70% ethanol for few minutes and then centrifuged for 2-3 min at room temperature. The supernatant was discarded and the pellet dried for approximately 1-2 hrs. The DNA was resuspended in 20 μL of sterile water and the quality and concentration was confirmed using a 0.8% agarose gel with ethidium bromide staining.

For the PCR reaction, 1 μL plant DNA (20-100 ng/μL) was combined with 17.8 μL serile water, 2.5 μL 10×Taq Buffer containing 15 'mM MgCl₂, 2,5 μL dNTPs (1 mM), 0.5 μL primer 1 (10 pmoles/μL). 0.5 μL primer 2 (10 pmoles/μL), and 0.2 μL Taq polymerase. The total volume of the reaction was 25 μL. The primer pairs used to detect the bar gene were;

| | |
|---|---|
| 5′-CCGTCTGCGGGAGCGCTATCC-3′ | (SEQ ID NO:1); and |
| 5′-CATCGCAAGACCGCCAACAGG-3′ | (SEQ ID NO:2). |

and the primer pairs used to detect the MnSOD gene were:

| | |
|---|---|
| 5′-AGAAACCAAAGGGTCCTG-3′ | (SEQ ID NO:3); and |
| 5′-GAGCAGACGGACCTTAGC-3′ | (SEQ ID NO:4). |

For amplification using the bar primer pairs, the PCR program was 5 min at 94° C., then 30 cycles of 94° C. for 1 min, 70° C. for 1.5 min and 72° C. for 1.5 min, followed by 5 min at 72° C. and holding at 4° C. (FIG. 2(A)). For amplification using The MnSOD primer pairs, the PCR program was 5 min at 94° C., then 30 cycles of 94° C. for 1 min, 56° C. for 1.5 min and 72° C. for 1.5 min, followed by 5 min at 72° C. and holding at 4° C. (FIG. 2(B)). PCR products were visualized on a 0.8% agarose gel with ethidium bromide.

Expression of the introduced MnSOD gene was detected using native PAGE gels. Leaf tissue was excised from a vegetative stage shoot. The sample (0.5 g) was frozen in liquid nitrogen, ground, and resuspended in 1 ml of 50 mM KH₂PO₄, pH 7.8. The homogenate was centrifuged at 13,000×g for 15 min at 4° C., the supernatant was transferred to a clean rube, and centrifuged again for 5 min at 4° C. The protein content of the supernatant was determined (Coomasie Protein Assay, Pierce). A constant volume (20 μL) containing 150 μg protein was applied w a 13% polyacrylamide gel with a 4% stacking gel (McKersie et al. 1993 Plant Physiology 103:1155-1163). The proteins were separated at 10 mA constant current for 1 h, and 15 mA for the following 2 h The gel was stained for 20 to 30 min in dark at 4° C. with an equal volume mix of staining solution A (0.06 nM riboflavin, 0.651% TEMED in 100 ml phosphate buffer (50 mM KH₂PO₄ and 50 mM KH₂HPO₄)) and solution B (2.5 mM⁻NBT in 100 ml phosphate buffer). Then the gel was illuminated at 4° C. in a light box for 20 min. Areas of superoxide dismutase activity were negatively stained against a blue background.

An experiment was conducted to evaluate the vacuum treatment during exposure of the tissue to Agrobacterium, and to evaluate the effect of maintaining the explant tissue on differentiation medium in the absence of selection agent for a four-week period following exposure to Agrobacterium. This experiment involved the following treatment combinations:

A) the transformation protocol as outlined above, with vacuum during Agrobacterium exposure;

B) a control treatment with no Agrobacterium during the vacuum treatment;

C) the transformation protocol as outlined but without vacuum during the Agrobacterium exposure;

the transformation protocol of (A), however, the length of time prior to transfer to a medium containing the selection agent (i.e. To produce differentiated calli) is

D) 3 weeks,

E) 2 weeks;

F) 1 week; and

G) 0 week,

The explants in the last treatment were immediately transferred to selection medium following Agrobacterium exposure. In all treatment combinations, the row length of time the explants were cultured in the dark was 4 weeks. For example, treatment F (1 week treatment) involved 1 week dark, incubation on selection agent free MSCB medium, 3 weeks dark incubation on MSCB medium containing the selection agent, followed by transfer to MSO medium containing the selection agent and cultured in the light. Table 2 summarizes the results of tis experiment.

Table 2 Summary of a transformation experiment comparing the vacuum treatment during Agrobacterium exposure, and the effect of reducing the number of weeks between the exposure of the explants to Agrobacterium and transfer to medium containing the selection agent.

TABLE 2
Summary of a transformation experiment comparing the vacuum
treatment during Agrobacterium exposure, and the effect of reducing the number
of weeks between the exposure of the explant to Agrobacterium and transfer to
medium containing the selection agent.

| | | No. weeks | | | |
|---|---|---|---|---|---|
| | | with no | | No. of embryogenic | % |
| | | selection | No. | explants following | Transformation |
| Treatment | Vacuum | agent | explants | selection in light | efficiency |
| A. Agrobacterium | yes | 4 | 24 | 18 | 75% |
| B. No- | yes | 4 | 10 | 0 | 0% |
| Agrobacterium | | | | | |
| C. Argobacterium | NO | 4 | 26 | 11 | 42% |
| D. Agrobacterium | yes | 3 | 23 | 9 | 39% |
| E. Agrobacterium | yes | 2 | 23 | 1 | 4% |
| F. Agrobacterium | yes | 1 | 25 | 0 | 0% |
| G. Agrobacterium | yes | 0 | 23 | 0 | 0% |

Applying the transformation method as described in the present invention, 75% of the explants generated embryos following stepwise transfer on media containing the selection agent. As expected, the no-Agrobacterium control (B) did not produce embryos on media containing the selection agent. Nor applying the vacuum treatment (C) during Agrobacterium exposure reduced the transformation efficiency from 75% to 42%. Decreasing the time between Agrobacterium exposure and transfer to medium containing the selection agent reduced The transformation efficiency; a 3 week period reduced the efficiency to 39% and a 2-week period reduced he efficiency To 4%. No embryos were obtained if the period was reduced to 1-week or if the explants were immediately transferred to medium containing the selection agent.

### Example 2

### Transformation of Perennial Ryegrass

Perennial ryegrass (Lolium perenne L.) was transformed using the above method with a binary vector. The DNA construct was that used in Example 1.

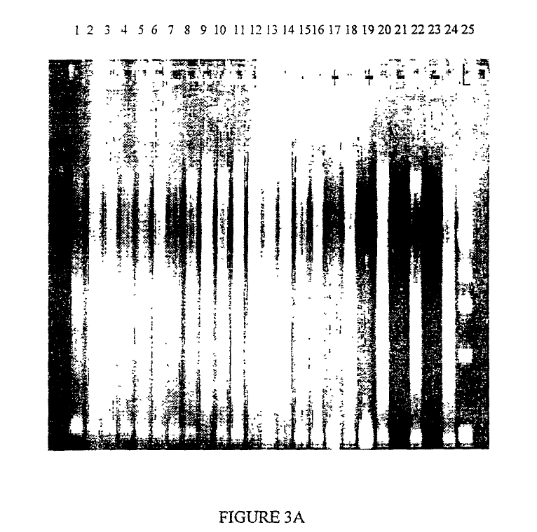

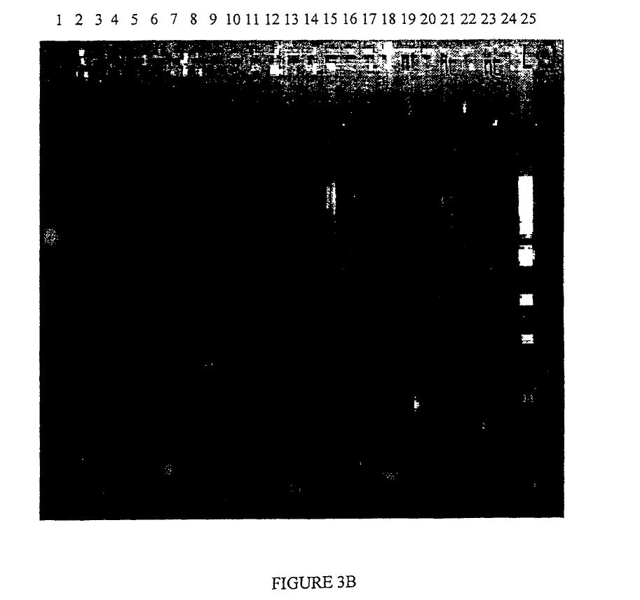

To obtain cultured explant tissue, seeds were surface sterilized, germinated for 3 days in the dark. Seedlings were surface sterilized and transferred to solid embryogenic callus induction medium (MSPR) and maintained in the dark for 10-60 days. The construct was introduced via Agrobacterium mediated transformation as outlined above using MSPRcc (liquid) co-cultivation medium. Following the 3 day co-cultivation, the explants were ten placed on MSPR solid medium containing 300-500 mg/L claforan and cultured in the dark from about 22 to about 25° C. for 3-8 wk. Transformed calli were transferred to MSO medium containing the selection agent PPT (phosphinothricin) at 5 mg/L and cultured in the light to allow the plants to grow. After 4 wk, surviving calli were transferred to MSO medium containing 5 mg/L PPT, and after an additional 2 wk. were transferred to MSO medium containing 10 mg/L PPT. Through the selection phase, all cultures were maintained in the light. Plants recovered from this process were sampled for the presence of the introduced genes using PCR (see FIG. 3). DNA was extracted from putative transgenic perennial ryegrass plants using a method from CIMMYT (Applied Molecular Genetics Laboratory Manual p.2-3) with the following thee modifications; 600 μL CTAB at room temperature was added to approximately 20 5 mm pieces of grass tissue, the samples were homogenized using a Fast Prep FP120 machine (Bio 101) for 20 seconds then heated at 68 ° C. for 20 min, and 4-5 μL RNAase A (10 ng/μL) was used in the reaction mixture. Approximately 20 ng of genomic DNA was then used in a PCR reaction (Stratagene Robocycler 96) to amplify the bar gene (468 bp product), with 1.3 units Expand High Fidelity DNA polymerase mix (Roche Diagnostics), 0.1 mM dNTP, 1/10 10×Expand HF buffer with 15 mM MgCl₂, and 0.2 μM of each primer 1 and primer 2 as described for creeping bentgrass. The amplification was a single 5 minute cycle at 94° C. followed by 30 cycles of 1.5 minutes at 94° C., 2 minutes at 68° C. and 2 minutes at 72° C. and ended with one 5 minute cycle at 72° C. Each PCR sample was electrophoresed on a 0.8%/TBE agarose gel and visualised by staining with ethidium bromide.

Table 3 summaries the results of four separate transformation experiments conducted using two varieties of Perennial ryegrass. Elite and Affinity. The transformation efficiency, computed as the proportion of explants that Transgenic plants were recovered from and transferred Lo a greenhouse, averaged 25% with a range of 10-55% among the four experiments and for the two varieties.

TABLE 3
Summary of transformation experiments conducted
using embryogenic calli of Perennial ryegrass.

| | | | Total | |
|---|---|---|---|---|
| | | | number of | |
| | | No. of | putative | |
| | | explants | transgenic | |
| | No. of | following | plants | PCR Screening, |
| Variety | explants | selection | recovered | % plants positive |
| Elite | 40 calli | 5 | 4 | 100 |
| Elite | 20 calli | 11 | 21 | 100 |
| Affinty | 20 calli | 6 | 4 | 100 |
| Elite | 100 calli | 13 | 169 | 89 |

| | | |
|---|---|---|
| Transformation | Mean | 25% |
| efficiency (% of | | |
| explants yielding | | |
| transgenic | Range | 10-55% |
| plants) | | |

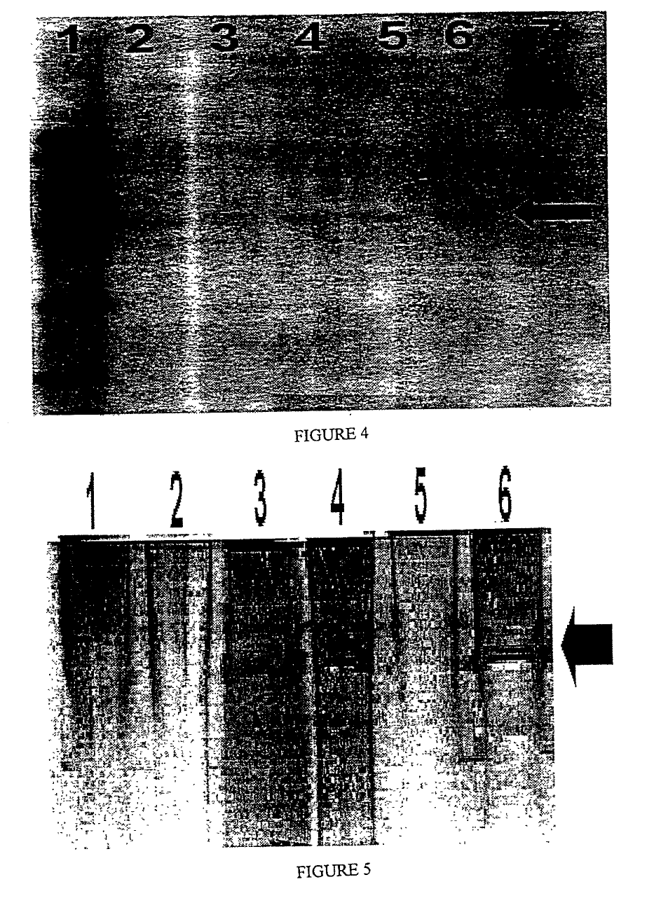

Southern hybridization was performed to confirm genomic insertion of the gene construct (FIG. 4). Genomic DNA was extracted using the method of Junghans and Metzlaff (1990 BioTechniques 8:176) wit an extra step to precipitate polysaccharides as described by Michales et at., 1994 (BioTechniques 17:274-276). Briefly, after phenol/chloroform extraction, NaCl was added to a concentration of 25 mM. 0.35 volume of 100% ethanol at room temperature (RT) was added dropwise with continual mixing and samples were incubated on ice for 20 min. Samples were spun at 10,000×g for 5 min, the supernatant was transferred to a fresh rube and precipitated with 1 volume of isopropanol. DNA concentration was determined using a specrofluorophotomerer (Shimadzu) and the Hoechst dye assay (Hoefer Scientific Instruments Technical Bulletin #119). Hoechst 33258 dye was diluted to a final concentration of 0.1 μg/mL in 1×TNE (10 mM Tris, 1 mM EDTA, 0.1 M NaCl, pH 7.4). 2 μL of DNA was added and assayed at λ excitation 365 nm and μ emission 455 nm. DNA quality was also assessed by electrophoresis on a 0.8%/TBE agarose gel stained with ethidium bromide. A 15-25 μg DNA sample was digested overnight in a 400 μL restriction enzyme digest. After digestion, samples were precipitated with 2.5 volume of 100% ethanol and 0.1 volume of 3 M sodium acetate pH 7, both at 4° C., incubated at −20° C. for a minimum 1.5 hr, spun at 12,000× at 4° C. The pellet was washed 2× with 70% ethanol at 4° C. dried and resuspended overnight at 4° C. in 18 μL sterile ddH₂O, pH 8. DNA was loaded and electrophoresed on a 1% agarose/TBE gel at 20 volts overnight. 30 ng of a DIG labelled ladder and 500 pg of pSODbar plasmid was also loaded. DNA was transferred to a positively charged nylon membrane (Roche Diagnostics) by capillary transfer with 20×SSC buffer overnight at RT. Following the transfer, the membrane was UV crosslinked on both sides, rinsed briefly in ddH₂O and allowed to dry completely.

The membrane was prehybridized in DIG Easy Hyb Solution (prepared from DIG Easy Hyb Granules, Roche Diagnostics) for 30 mm at 37° C. PCR DIG Probe Synthesis Kit was used to prepare a Dieoxigenin-labelled bar probe by PCR of 200 pg of pSODbar using the primers P1 (SEQ ID NO: 1) and P) (SEQ ID NO; 2) and conditions described above, except that the final nucleotide concentrations were 2mM aATP, dCTP, dGTP, 1.3 mM dTTP and 0.7 mM DIG-dUTP (Roche Diagnostics). The PCR labelled probe was diluted 1 μL 1 mL with DIG Easy Hyb solution. The probe was denatured prior TO use by hearing at 68° C. for 10 min and flash cooling on ice. Following prehybridization. The membrane was incubated overnight at 37° C. with the DIG probe. The membrane was washed 2×5 min in 2×SSC (0.1% SDS) at RT, followed by 2×15 min. washes in 0.5×SSC (0.1% SDS) at 68° C. Blocking and detection were carried out as described by the manufacturer (Roche Diagnostics) using the DIG Wash and Block Buffer Set, Anti-Digoxigenin AP Fab Fragments and CDP-Star chemiluminescent substrate. The membrane was exposed to X-Omat AR film (Kodak) until The desired signal was attained (FIG. 4).

To confirm that the introduced MnSOD gene was expressed, Western blot hybridization was performed (FIG. 5). Total protein was extracted from young leaf blade tissue trough vortexing with liquid nitrogen in 15 mL Nalgene tubes including 3 cylindrical grinding beads (Bio 101). Frozen homogenized tissue (100 mg) was added to 125 μL of KH₂PO₄ buffer pH 7.8 on ice. Samples were centrifuged at 4° C. at 12,000×g for 10 min. The supernatant was removed and spin repeated. Protein concentration was determined spectrophotometrically using the Pierce Coomassie Plus Protein Assay Reagent Kit (Pierce). Samples were diluted 1:1 with sample buffer and 50 μg of protein for each sample were loaded onto a 0.75 mm thick non-denaturing polyacrylamide get consisting of 4% stacking and 12% separating sections. The proteins were electrophoresed at 4° C. in a Mini Protean II system (Bio-Rad) at 550 volts with 7.5 mA per gal while stacking and 15 mA per gel while separating in rank buffer composed of 25 mM Tris and 64 mM L-isoleucine. Electrophoresis was stopped when the dye front ran off the bottom of the gel. The gel was then equilibrated for 15 min at RT in transfer buffer composed of 25 mM Tris and 192 mM glycine. Proteins were electroblotted overnight at 4° C. onto an Immuno-Blot PVDF membrane (Bio-Rad) pre-wet in 100% methanol and equilibrated in transfer buffer for 5 min. A Mini Trans-Bloc cell (Bio-Rad) was used for electroblotting at 30 volts and 90 mA. Voltage was increased to 100 for 30 min in the morning, then the membrane was rinsed 3×5min at RT in sterile ddH₂O and allowed to cry completely. Immunologic detection was carried our following the manufacturers instructions (Bio-Rad Amplified Opti-4CN Detection Kit) except that the blocking solution was 5% non-fat dry milk powder in Tris Buffered Saline (TBS) composed of 20 mM Tris pH 7.5 with HCl and 500 MM NaCl. The membrane was incubated for 1.5 hr in antibody against MnSOD (diluted 1:5000) from N. plumbaginifolia provided by Laboratorium voor Genetica, Universiteit Gent, Gent, Belgium which were prepared as described by Bowler et al.991 (EMBO J. 10:1723-1732). The membrane was then incubated for 1.15 hr with goat-ant rabbit antibody diluted 1:3000 (FIG. 5).

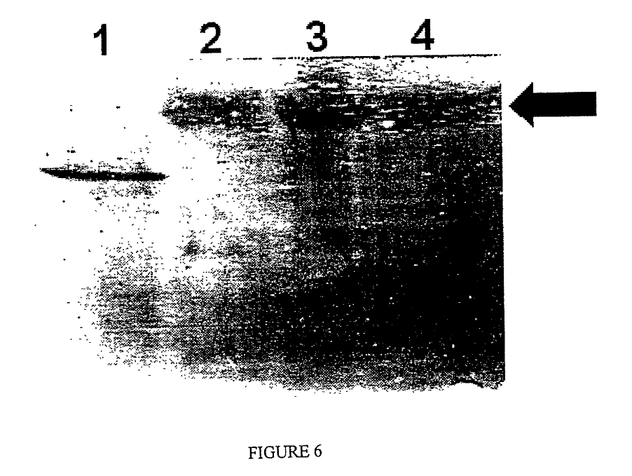

To confirm that the introduced MnSOD gene produced an active protein, SOD enzymatic activity was measured using native PAGE gels. Total protein was extracted and electrophoresed as described above, except that 300 μg of protein were loaded onto the gel which was composed of 4% stacking and 10% separating sections. Following electrophoresis, the gel was assayed for SOD activity in a stain containing 0.06 mM riboflavin, 0.651% N,N,N′,N′-Tetramethylethylenediamine (TEMED and 2.5 mM Nitro Blue Tetrazolium (NBT) in phosphate buffer (50 mM K₂PO₄pH adjusted to 7.8 with 50 mM KH₂PO₄) for 30 min in the dark at 4° C. In order to stain specifically for MnSOD activity, Cu/ZnSOD and FeSOD isozymes were inactivated by 30 min incubation act RT in 3% H₂O₃ in phosphate buffer, followed by staining for SOD activity as described. Gels were illuminated at 4° C. on a white light box for approximately 20 minutes, or until the desired signal was attained (FIG. 6).

Treatment with 3% H₂O₂, inhibited the activity of Cu/Zn and FeSOD isozymes. Following the treatment, areas of MnSOD activity were apparent on the gel (FIG. 6). Transgenic plants had greater MnSOD activity and a unique isozyme band compared to extracts from non transgenic plants. This confirmed that the introduced MnSOD transgene expressed an active MnSOD enzyme in these transgenic plants.

### Example 3

### Transformation of Perennial Ryegrass Mature Zygotic Embryos

Perennial ryegrass seeds were dehusked, surface sterilized and allowed to germinate on sterile filter paper overnight. Embryos were then dissected from the seeds and embryos were placed in a 1.5 mL microfuge tube (20 embryos per tube) and were transformed with Agrobacterium as outlined above using MSPRcc (liquid) co-cultivation medium. Following the vacuum treatment, the rubes were sealed and placed horizontally for the remainder of the 1 hour incubation period. Following incubation, all liquid was pipetted from the tubes and 1.5 mL of MSPRcc (liquid) co-cultivator medium was added. Embryos were incubated, horizontally, in The dark for 3 days. Embryos were rinsed, blotted dry, and were plated to MSPR solid embryo induction medium for callus induction for 4 weeks in the dark and transferred to fresh MSPR for an additional 4 weeks in the dark Transformed calli were then transferred to MSO medium containing the selection agent PPT (phosphinothricin) at 5 mg/L and cultured in the light to allow the plans to grow After 4 wk. surviving calli were transferred to MSO medium containing 5 mg/L PPT, and after an additional 2 wk. were transferred to MSO medium containing 10 mg/l PPT. Through the selection phase, all cultures were maintained in the light. Plants recovered from this process were sampled for the presence of the introduced genes using PCR. Leaf tissue was excide and DNA extracted using a Qiagen Dneasy Plant Mini Kit (Qiagen Inc., Mississauga, ON) following the manufacturer's instructions. The DNA extract was subjected to PCR using the same protocol and primer pairs as described for creeping bentgrass (Example 1).

The following cable (Table 4) summaries the results of two transformation experiments conducted using dissected embryos of Perennial ryegrass The transformation efficiency computed as the percent explants that formed embryos on selection medium averaged 22.5%, similar to that found for previous experiments using calli as an explant source (Example 2).

Table 4
Summary or transformation experiments conducted using embryos of
Perennial ryegrass.

| | | No. of embryogenic | |
|---|---|---|---|
| | No. of | explants | PCR Screening. % |
| Variety | explants | following selection | plants positive |
| Elite | 40 embryos | 8 | 100 |
| Elite | 40 embryos | 10 | 100 |
| Transformation | Mean | 22.5% | |
| efficiency* | Range | 20-25% | |

*% of explants yielding transgenic plants

All citations are incorporated by reference.

The present invention has been described with regard to preferred embodiments. However, it will be obvious to persons skilled in the art that a number of variations and modifications can be made without departing from the scope of the invention as described herein.

1. A method for the transformation of a monocot plant comprising,
i) exposing explant tissue of said monocot plant to an Agrobacterium strain under vacuum in the presence of a phenolic compound, said Agrobacterium strain comprising a heterologous gene of interest within a vector;
ii) removing said Agrobacterium from said explant tissue;
iii) adding an antibiotic against said Agrobacterium; and
iv) selecting explant tissue for occurrence of said heterologous gene of interest.

2. The method of claim 1 wherein said step of selecting explant tissue further comprises:
i) maintaining said explant tissue on media in absence of a selection agent producing differentiated calli;
ii) transferring said differentiated calli to media containing a selection agent; and
iii) obtaining calli that grow in the presence of the selection agent.

3. The method of claim 1 wherein said Agrobacterium comprises a regular binary vector.

4. The method of claim 1 wherein said explant tissue is callused coleoptile node.

5. The method of claim 1 wherein said explant tissue comprises a zygotic embryo.

6. The method of claim 2 wherein said Agrobacterium comprises a super virulent vector.

7. The method of claim 1, wherein said phenolic compound is acetosyringone.

8. The method of claim 7, wherein said explant tissue is exposed under vacuum from about 10 to about 15 min.

9. The method of claim 8, wherein said antibiotic comprises claforan.

10. A method for the transformation of a monocot plant comprising,
i) placing explant tissue of said monocot plant into media comprising a suspension of Agrobacterium to obtain a mixture, said Agrobacterium strain comprising a heterologous gene of interest within a vector;
ii) maintaining said mixture under vacuum in the presence of acetosyringone;
iii) releasing said vacuum and further incubating said explant tissue in the presence of said Agrobacterium;
iv) transferring said explant tissue to fresh media comprising acetosyringone and incubating said explant tissue in the dark
v) washing said explant tissue with an antibiotic against said Agrobacterium,
vi) transferring said explant tissue to fresh media and allowing said explant tissue to differentiate, thereby producing differentiated calli;
vii) placing said differentiated calli onto media containing a selection agent, and maintaining said differentiated calli in the light; and
viii) obtaining calli that grow in the presence of the selection agent.

11. The method of claim 10 wherein said Agrobacterium comprises a super virulent vector.

12. The method of claim 10 wherein said Agrobacterium comprises a regular binary vector.

13. The method of claim 10 wherein the fresh media of step v) comprises said antibiotic against said Agrobacterium.

14. The method of claim 10 wherein said explant tissue is callused coleoptile node.

15. The method of claim 10 wherein said explant tissue comprises a zygotic embryo.

16. The method of claim 13 wherein said antibiotic is claforan.

17. A method for the transformation of a monocot plant comprising,
i) placing explant tissue of said monocot plant into media comprising a phenolic compound, and a suspension of Agrobacterium to obtain a mixture, said Agrobacterium strain comprising a heterologous gene of interest within a vector;
ii) washing said explant tissue with an antibiotic against said Agrobacterium and transferring said explant tissue to fresh media comprising acetosyringone and incubating said explant tissue in the dark;
iii) transferring said explant tissue to fresh media and allowing said explant tissue to differentiate, thereby producing differentiated calli;
iv) placing said differentiated calli to media containing a selection agent, and maintaining said differentiated calli in the light; and
v) obtaining calli that grow in the presence of the selection agent.

18. The method of claim 17 wherein said Agrobacterium comprises a super virulent vector.

19. The method of claim 17 wherein said Agrobacterium comprises a regular binary vector.

20. The method of claim 17 wherein the media of step iii) comprises said antibiotic against said Agrobacterium.

21. The method of claim 17 wherein said explant tissue is callused coleoptile node.

22. The method of claim 17 wherein said explant tissue comprises a zygotic embryo.

23. The method of claim 2, wherein, in said step of maintaining, said explant tissue is maintained on media in absence of a selection agent from about 3 weeks to about 8 weeks.

24. The method of claim 10, wherein, in said step of transferring, said explant tissue is maintained on media in absence of a selection agent from about 3 weeks to about 8 weeks.

25. The method of claim 17, wherein, in said step of transferring, said explant tissue is maintained on media in absence of a selection agent from about 3 weeks to about 8 weeks.


SEQUENCE LISTING (4 sequences)

SEQ ID NO: 1 - DNA, 21 bp, ARTIFICIAL SEQUENCE
  misc_feature (1)..(21): PRIMER
ccgtctgcgggagcgctatcc

SEQ ID NO: 2 - DNA, 21 bp, ARTIFICIAL SEQUENCE
  misc_feature (1)..(21): PCR primer
catcgcaagaccggcaacagg

SEQ ID NO: 3 - DNA, 18 bp, Artificial sequence
  misc_feature (1)..(21): PCR primer
agaaaccaaagggtcctg

SEQ ID NO: 4 - DNA, 18 bp, Artificial sequence
  misc_feature (1)..(21): PCR primer
gagcagacggaccttagc